(19) United States
(12) Patent Application Publication
(10) Pub. No.: US 2013/0149250 A1
(43) Pub. Date: Jun. 13, 2013

(54) HYPERPOLARIZED AGENTS FOR MRI CHARACTERIZATION OF REDOX SYSTEMS IN VIVO

(71) Applicants: David M. WILSON, San Francisco, CA (US); Kayvan KESHARI, Stockton, CA (US); John KURHANEWICZ, San Francisco, CA (US); Daniel VIGNERON, San Francisco, CA (US)

(72) Inventors: David M. WILSON, San Francisco, CA (US); Kayvan KESHARI, Stockton, CA (US); John KURHANEWICZ, San Francisco, CA (US); Daniel VIGNERON, San Francisco, CA (US)

(73) Assignee: The Regents of the University of California, Oakland, CA (US)

(21) Appl. No.: 13/532,692

(22) Filed: Jun. 25, 2012

Related U.S. Application Data
(60) Provisional application No. 61/500,556, filed on Jun. 23, 2011.

(51) Int. Cl. C07D 307/33

(52) U.S. Cl. CPC C07D 307/33
USPC 424/9.3; 549/315; 435/29; 435/4

(57) ABSTRACT

The present invention provides a MRI probe of use in detecting and characterizing redox systems in vivo. Also provided are methods of using the probe in MR imaging experiments for diagnosis of disease in a subject, for drug discovery and for probing the redox states of biological systems in vitro.

### CROSS REFERENCE TO RELATED APPLICATIONS

This application claims the benefit of priority under 35 U.S.C. §119(e) to U.S. Application No. 61/500,556, filed Jun. 23, 2011 and is hereby incorporated by reference as though fully set forth herein.

### GOVERNMENT RIGHTS

This invention was made with government support under R21EB005363, R01 EB007588 awarded by National Institutes of Health. The government has certain rights in the invention.

### BACKGROUND OF THE INVENTION

Magnetic resonance imaging is a particularly attractive diagnostic technique as it is non-invasive and does not involve exposing the patient under study to potentially harmful radiation such as X-rays.

In order to achieve effective contrast between MR images of different tissue types, MR contrast agents (e.g. paramagnetic metal species) are administered to the subject undergoing imaging. MR contrast agents affect relaxation times in the zones in which they are administered or at which they concentrate.

The MR sensitive nuclei are characterized by different spin states. MR signal strength is dependent on the population difference between the nuclear spin states of the imaging nuclei, which is governed by a Boltzmann distribution dependent on both temperature and magnetic field strength.

Techniques have been developed which involve ex vivo nuclear spin polarization of agents containing non-zero nuclear spin nuclei (e.g., ³He) prior to administration and MR signal measurement. Some such techniques involve the use of polarizing agents, for example, conventional OMRI contrast agents or hyperpolarized gases to achieve ex vivo nuclear spin polarization of non-zero nuclear spin nuclei in an administrable MR imaging agent.

The use of hyperpolarized MR contrast agents in MR investigations such as MR imaging has the advantage over conventional MR techniques in that the nuclear polarization to which the MR signal strength is proportional is essentially independent of the magnetic field strength in the MR apparatus. Currently the highest obtainable field strengths in MR imaging apparatus are about 8 T, while clinical MR imaging apparatus are available with field strengths of about 0.2 to 1.5 T. Since superconducting magnets and complex magnet construction are required for large cavity high field strength magnets, these are expensive. When using a hyperpolarized contrast agent, it is possible to make images at all field strengths from earth field (40-50 μT) up to the highest achievable fields because the field strength is less critical. However there are no particular advantages to using the very high field strengths where noise from the patient begins to dominate over electronic noise (generally at field strengths where the resonance frequency of the imaging nucleus is 1 to 20 MHz). Accordingly, the use of hyperpolarized contrast agents opens the possibility of high performance imaging using low cost, low field strength magnets.

Reduction and oxidation (redox) chemistry is involved in both normal and abnormal cellular function, and in processes as diverse as circadian rhythms and neurotransmission. Intracellular redox is maintained by coupled reactions involving NADPH, glutathione (GSH), and vitamin C, as well as their corresponding oxidized counterparts. In addition to functioning as enzyme cofactors, these reducing agents have a critical role in dealing with reactive oxygen species (ROS), the toxic products of oxidative metabolism seen as culprits in aging, neurodegenerative disease, and ischemia/reperfusion injury. Despite this strong relationship between redox and human disease, there is no known method to interrogate a redox pair in vivo.

Although redox chemistry is a central feature of cellular processes, probes to interrogate coupled oxidation/reduction reactions in vivo are lacking. An MRI-detectable probe and methods for MRI analysis of redox systems utilizing such a probe would represent a significant advance in the diagnosis of disease and in fields in which in vitro investigation and characterizations of redox systems is of use.

### BRIEF SUMMARY OF THE INVENTION

The present invention provides the first instance of a probe for in vivo MR imaging of a one or more member of a redox couple. In various embodiments, the probe comprises a molecular structure capable of reduction, oxidation or both in a redox active system. The probe also includes as a component of its structure an atom that is both detectable in an MR experiment and capable of being hyperpolarized. In exemplary probes, the compound is enriched with respect to the hyperpolarizable atom.

In an exemplary embodiment, the invention provides an MRI-detectable, redox sensitive probe based on [1-¹³C] dehydroascorbate [DHA], the oxidized form of Vitamin C. This probe serves as an endogenous redox sensor for in vivo imaging using hyperpolarized ¹³C spectroscopy. In murine models, hyperpolarized [1-¹³C] DHA is rapidly converted to [1-¹³C] vitamin C within the liver, kidneys, and brain, as well as within tumor in a transgenic prostate cancer (TRAMP) mouse. This result is consistent with what has been previously described for the DHA/Vitamin C redox pair, and indicates a role for hyperpolarized [1-¹³C] DHA in characterizing the redox state of tumors.

The present invention provides probes and methods for using such probes and methods determining vulnerability of both normal and abnormal tissues to ROS.

### BRIEF DESCRIPTION OF THE DRAWINGS

FIG. 1. Biochemical mechanism, hyperpolarization and reduction of [1-¹³C] DHA. (a) Relationship between redox pairs NADPH/NADP, GSSG/GSH and Vitamin C/DHA with associated enzymes. (b) Reversible reduction of labeled DHA to Vitamin C demonstrating the position of the hyperpolarized carbon. (c) Representative decays of hyperpolarized [1-¹³C] DHA and [1-¹³C] Vitamin C at 3 T. Spectra were acquired using a 5 degree pulse with 3 second temporal resolution. (d) To validate the reduction, 2.5 mM hyperpolarized DHA was reacted with increasing concentrations of NaCNBH₃. VitC—Vitamin C. VitC/Total—Vitamin C/Total ¹³C signal.

FIG. 2. Real-time dynamics demonstrating the in vivo reduction of DHA to Vitamin C. (a) Coronal T₂-weighted image of a normal mouse demonstrating the region of excitation for dynamic experiments. (b) Representative dynamic spectra post-injection of 350 uL of 15 mM hyperpolarized [1-¹³C] DHA. In vivo spectra were acquired using a 5 degree pulse with 3 second temporal resolution at 3 T. An 8M ¹³C-urea phantom was used as a chemical shift reference. (c) Spectra of each individual compound demonstrating the peak locations of DHA and Vitamin C (VitC) as compared to the dynamic data. (d) Average production of hyperpolarized Vitamin C in the dynamic spectra of normal mice (n=3). Error bars indicate±standard deviation of mean. VitC—Vitamin C. VitC/Total—Vitamin C/Total ¹³C signal.

FIG. 3. Coronal T₂-weighted images and corresponding ¹³C 3D MRSI demonstrating distribution of hyperpolarized DHA and Vitamin C (VitC) in a transgenic model of prostate adenocarcinoma (TRAMP) mouse.

FIG. 4. Axial T₂-weighted images and corresponding color overlays of hyperpolarized DHA and Vitamin C (VitC) signal in a normal rat brain.

FIG. 5. Scheme for synthesis, hyperpolarization and reduction of DHA inside of the cell.

FIG. 6. In vivo dynamics post-injection of hyperpolarized [1-¹³C] Vitamin C. (left) time-resolved ¹³C spectra were acquired using a 5 degree pulse with 3 second temporal resolution. (right) Sum of all spectra in the experiment, demonstrating the trace conversion of Vitamin C (VitC) to DHA.

FIG. 7. Hyperpolarization of Vitamin C and its oxidation to DHA in vivo.

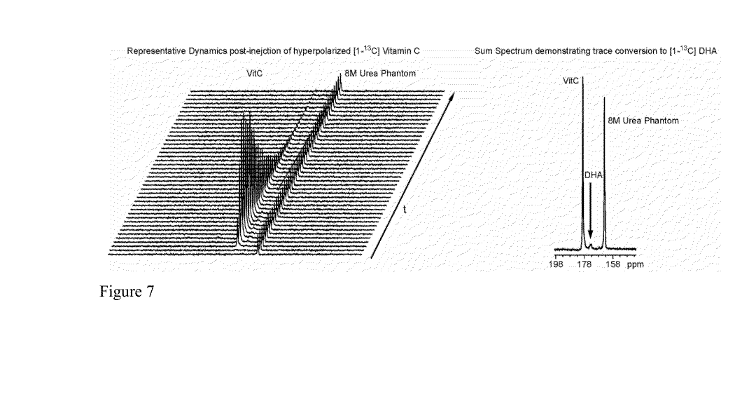

FIG. 8. In vivo 2D ¹³C MRSI studies in the normal rat brain. (a) HP [1-¹³C] DHA study demonstrating reduction to [1-¹³C] VitC within brain. No observable VitC signal was observed in voxels corresponding to surrounding tissue. (b) HP [1-¹³C] VitC study demonstrating no oxidation to [1-¹³C] DHA. The HP [1-¹³C] VitC signal is diminished in brain voxels, consistent with limited blood-brain-barrier penetration. (c) Average metabolite ratios calculated for brain voxels (n=16) following injection of HP DHA.

FIG. 9. (a) Representative high resolution T₂-weighted images and Lipid percentage images for a baseline and 2 week MCD mouse. (b) Hematoxylin and Eosin (H&e) and Oil Red staining demontrating the increase in lipid droplets at 2 weeks. (c) T₂-weighted images and corresponding hyperbolized ¹³C MR spectra post-injection of HP DHA, in a representative mouse at baseline and at 2 weeks of MCD diet. (d) Significant increase in VitC/VitC+DHA as well as VitC/DHA after 2 weeks on the MCD diet (P=0.01 and 0.005).

FIG. 10. (a) H&E—structure, Ki-67 nuclear antigen—proliferation and p-Imidazole (PIM)—hypoxia in a TRAMP tumor (b) Representative color overlay of ¹⁸FDG PET on CT and hyperpolarized (HP) DHA from the same TRAMP mouse (c) Ratio of Vitamin C (VitC) to total HP signal in TRAMP tumor and surrounding tissue (d) Comparison of signal ratio in tumors relative to surrounding tissue for ¹⁸FDG PET, HP VitC/VitC+DHA and HP Total ¹³C.

FIG. 11. Coronal T₂-weighted image of TRAMP and axial T₂-weighted image of a normal mouse prostate with corresponding hyperpolarized ¹³C MR spectra post-injection of HP DHA demonstrate elevated levels of vitamin C in TRAMP compared to the normal control.

FIG. 12. (a) Mercury orange staining of TRAMP and normal prostate demonstrate elevated levels of non-protein thiols; mean fluorescent intensity averaged over three ROIs was higher in TRAMPs than normal. (b) Intracellular glutathione levels were higher in TRAMP prostate compared to normal prostate. (c) Relative % expression of GLUT 1, 3, 4 transporters, glutathione-S-transferase, Na⁺ channel 1 and 2, protein disulfide isomerase, and thioredoxin reductase in TRAMP tumors versus normal prostate.

### DETAILED DESCRIPTION OF THE INVENTION

### Introduction

Reduction and oxidation (redox) chemistry is involved in both normal and abnormal cellular function, in processes as diverse as circadian rhythms and neurotransmission. Intracellular redox is maintained by coupled reactions involving NADPH, glutathione (GSH), and vitamin C, as well as their corresponding oxidized counterparts. In addition to functioning as enzyme cofactors, these reducing agents have a critical role in dealing with reactive oxygen species (ROS), the toxic products of oxidative metabolism seen as culprits in aging, neurodegenerative disease, and ischemia/reperfusion injury. Despite this strong relationship between redox and human disease, there is no known method to interrogate a redox pair in vivo. Broadly, the present invention provides MR-detectable, hyperpolarized compounds, which are new redox sensors. These new agents have a prognostic role in, inter alia, determining vulnerability of both normal and abnormal tissues to ROS.

### DEFINITIONS

“Subject” generally refers to a human. It also includes other mammals including those of the equine, porcine, bovine, feline, and canine families.

The term “compound” as used herein, is intended to include any solvates, hydrates, and polymorphs of any of the probes of the invention.

As used herein, the term “polarizing” refers to a procedure in which nuclei are temporarily significantly redistributed out of the ordinary population of energy levels. Hyperpolarization is accomplished using various techniques as described in recent review articles and book chapters (see, Kurhanewicz et al., (2008) J. Nucl. Med. 49(3), 341-344; Aime Set al., (2008) In Molecular Imaging, Handbook of Experimental Pharmacology. Vol 185/I. W. Semmler and M. Schwaiger (Eds). Springer-Verlag, Berlin, Germany. pp. 248-269; Ardenkjaer-Larsen et al., (2009) In Molecular Imaging: Principles and Practice. People's Medical Publishing House, Shelton, Conn. pp. 377-388; Bhattacharya P et al., Exp. Biol. Med. (Maywood) 234(12), 1395-1416; Brindle K (2008) Nat. Rev. Cancer 8, 1-14).

A “polarizing agent” is any agent suitable for performing ex vivo polarization of an MR imaging compound of the invention.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

As used herein, the term “polymorph” means solid crystalline forms of a compound or complex thereof which may be characterized by physical means such as, for instance, X-ray powder diffraction patterns or infrared spectroscopy. Different polymorphs of the same compound can exhibit different physical, chemical and/or spectroscopic properties. Different physical properties include, but are not limited to stability (e.g., to heat, light or moisture), compressibility and density (important in formulation and product manufacturing), hygroscopicity, solubility, and dissolution rates and solubility (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

The compounds of the present invention may contain one or more asymmetric carbon atoms. As such, a compound of this invention can exist as the individual “stereoisomers” (enantiomers or diastereomers) as well a mixture of stereoisomers. Accordingly, a compound of the present invention will include not only a stereoisomeric mixture, but also individual respective stereoisomers substantially free from one another stereoisomers. The term “substantially free” as used herein means less than 25% of other stereoisomers, preferably less than 10% of other stereoisomers, more preferably less than 5% of other stereoisomers and most preferably less than 2% of other stereoisomers, are present. Methods of obtaining or synthesizing diastereomers are well known in the art and may be applied as practicable to final compounds or to starting material or intermediates. Other embodiments are those wherein the compound is an isolated compound. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio.

“Isotopic substitution” refers to probes having an isotope in a greater amount than that isotope would be found in nature. Exemplary isotopes with which the compounds of the invention are substituted include, without limitation, ¹³C, ¹⁵N, ²H. Isotopic substitution in compounds of the invention can be accomplished by means known in the art.

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with an H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

The compounds of the invention can be further substituted with deuterium to yield probes having this isotope in an amount greater than its natural abundance. In an exemplary embodiment, synthesis of the compound of the invention includes the use of a deuterated precursor. Processes for de novo synthesis of deuterated compounds are known. See, for example, Yadav J S et. al., Adv. Synth. Catal. 2004 346: 77; Kirefu T, et. al., J. Label. Compd. Radiopharm. 2001 44: 329; Albrecht M, Synthesis 1996: 230; DePriest R N, U.S. Pat. No. 4,940,807). Other methods of substitution suitable for incorporation of deuterium are known to those of skill in the art of organic synthesis.

### The Embodiments

### Compositions

The present invention provides the first instance of a hyperpolarized probe for in vivo MR imaging of a one or more member of a redox couple. In various embodiments, the probe comprises a molecular structure capable of reduction, oxidation or both in a redox active system. The probe also includes as a component of its structure an atom that is both detectable in an MR experiment and capable of being hyperpolarized. In exemplary probes, the compound is enriched with respect to the MR-detectable hyperpolarizable atom.

In various embodiments, the hyperpolarizable atom is a member selected from ¹³C and ¹⁵N. In an exemplary embodiment, the hyperpolarizable atom is hyperpolarized in the compound of the invention.

In an exemplary embodiment, the probe of the invention is based on ascorbic acid (AA) or dehydroascorbic acid (DHA). In various embodiments, the compound includes at least one ¹³C atom. In various embodiments, the ¹³C is the carbon of the carbonyl moiety.

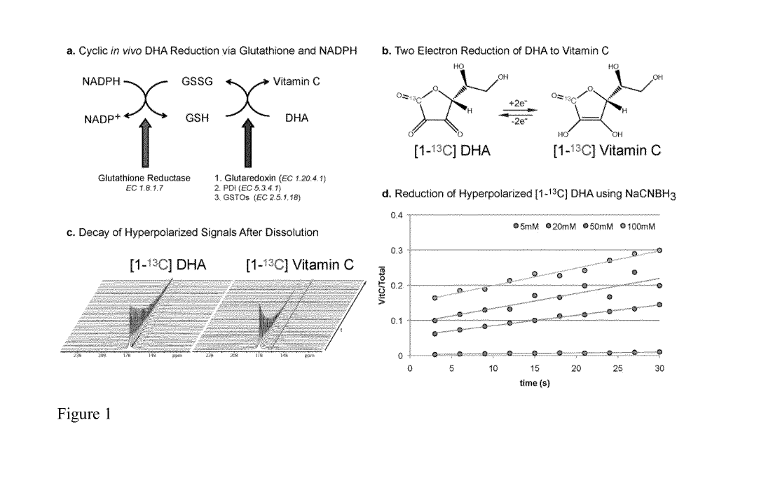

Ascorbic acid (Vitamin C) is an essential cofactor in reactions catalyzed by Cut dependent monooxygenases and Fe²⁺-dependent dioxygenases, required for the enzymatic biosynthesis of collagen, catecholamines, and peptide neurohormones. It is also an effective reducing agent protecting cells from the injurious effects of reactive oxygen species. Pauling championed the antiviral properties of Vitamin C, and cells of the immune system including mononuclear leukocytes and neutrophils are known to accumulate large quantities of ascorbate, which may be protective against free radicals generated during the respiratory burst. In reactions with free radicals, ascorbic acid acts as a stable electron donor, converted to the radical anion semidehydroascorbate, which can be recycled back to ascorbate by a number of mechanisms, or undergo disproportionation to form ascorbate and dehydroascorbate. Recently, a number of mechanisms have been elucidated for the two-electron reduction of DHA to Vitamin C in animals. This conversion can take place via a glutathione (GSH)-dependent mechanism catalyzed by glutaredoxin, protein disulfide isomerase, glutathione transferases, or by NADPH-dependent mechanisms including reduction catalyzed by 3α-hydroxysteroid dehydrogenase. In turn, GSH levels are maintained by the NADPH-dependent reduction of oxidized glutathione (GSSG), with the GSSG/GSH couple serving as an important indicator of the cellular redox environment (FIG. 1a). Intracellular redox is broadly linked to processes both normal and pathologic, but has particular relevance to cancer with glutathione metabolism playing both protective and pathologic roles. Loss of the glutathione S-transferase M1 gene (GSTM1) correlates with increased susceptibility to lung and bladder cancer, while elevated GSH in several tumors may confer resistance to chemo- and radio-therapy. These effects are thought to be secondary to cellular resistance to reactive oxygen species (ROS) including hydrogen peroxide and superoxide.

In an exemplary embodiment, there is provided a DHA-based probe which is rapidly converted to [1-¹³C] Vitamin C in vivo, at levels suitable for high-resolution magnetic resonance spectroscopic imaging (MRSI) (FIG. 1b). DHA is maintained at much lower intracellular concentrations than Vitamin C, but has a number of unique properties that set it apart. While the Nat dependent cotransporters SVCT1 and SVCT2 transport Vitamin C, DHA is transported by facilitated diffusion via the glucose transporters GLUT1, GLUT2, and GLUT4, with rapid reduction of DHA to Vitamin C occurring within the cell. The transport and structural properties of DHA make it an excellent candidate for in vivo metabolic studies using hyperpolarized ¹³C MRSI.

In various embodiments, the agent has a T₁ that is of sufficient length to allow imaging to be performed while a detectable fraction of the agent is still hyperpolarized. In an exemplary embodiment, the invention provides nuclear spin polarized MR imaging agents comprising in their molecular structure nuclei capable of emitting MR signals in a uniform magnetic field (e.g., MR imaging nuclei such as ¹³C or ¹⁵ N nuclei), which are also capable of exhibiting a long T₁ relaxation time, and preferably additionally a long T₂ relaxation time. Such agents are referred to hereinafter as “high T₁ agents”. A high T₁ agent, a term which, for the purpose of this application, does not include ¹H₂O, is water-soluble and has a T₁ value of at least 6 seconds in D₂O at 37° C. and at a field of 3 T, e.g., 8 s or more, e.g., about 10 s or more, e.g., about 15 s or more, e.g., about 30 s or more, e.g., about 70 s or more, or event about 100 s or more.

In an exemplary embodiment, the agent is hyperpolarized [1-¹³C] DHA, having a T₁ of at least about 57 s at a clinically relevant field strength (3 T), and facile chemical reduction to [1-¹³C] Vitamin C by NaBH₃CN with a 3.8 ppm downfield chemical shift.

In an exemplary embodiment, conversion of the probe to another member of the redox couple is observed in one or more organ system in vivo. For example, the rapid conversion to [1-¹³C] Vitamin C of [1-¹³C] DHA was observed in kidneys, liver, and tumor in a transgenic adenocarcinoma of the mouse prostate (TRAMP) model, as well as in a normal rat brain. These results highlight the utility of hyperpolarized [1-¹³C] DHA as a probe for redox chemistry in living biologic systems, and provide evidence that GSH-mediated reduction of DHA is a feature of prostate cancer that may be exploited therapeutically.

Unless the MR imaging nucleus is the naturally most abundant isotope, the molecules of a high T₁ agent will preferably contain the MR imaging isotope in an amount greater than its natural isotopic abundance (i.e. the agent is “enriched” with the isotope).

In exemplary embodiments, the isotope with which the compound of the invention is enriched (e.g., ¹⁵N or ¹³C nuclei) having a long T₁ relaxation time. Exemplary compounds include ¹³C enriched MR imaging agents having ¹³C at one particular position (or more than one particular position) in an amount in excess of the natural abundance, i.e., above about 1%. In exemplary compounds of the invention, each isotopically enriched carbon position will have 5% or more, 10% or more, 25% or more, 50% or more or even in excess of 99% (e.g., 99.9%) of ¹³C.

In various compounds of the invention, the ¹³C nuclei amount to >2% of all carbon atoms in the compound. The MR imaging agent is preferably ¹³C enriched at one or more carbonyl or quaternary carbon positions. In exemplary embodiments, the ¹³C nucleus in a carbonyl group or in certain quaternary carbons has a T₁ relaxation time of more than about 2 s, more than about 5 s, or even more than 30 s.

Exemplary ¹³C enriched compounds of the invention are those in which the ¹³C nucleus is immediately adjacent to and covalently bonded to one or more non-MR active nuclei such as ¹⁶0, ³²S, ¹²C. In other embodiments, the MR active nucleus is adjacent to and covalently bound to the carbon of a C═C double bond.

In various embodiments, the compound of the invention includes a hyperpolarizable, MR-detectable atom which is selected from ¹³C, ²H and a combination thereof.

### Pharmaceutical Formulations

In an exemplary embodiment, the invention provides a pharmaceutical formulation appropriate for administration to a subject in the course of an imaging experiment. The formulation includes a hyperpolarized compound of the invention and a pharmaceutically acceptable diluent.

The carrier(s) must be “acceptable” in the sense of being compatible with the other ingredients of the formulation and not deleterious to the recipient thereof. The term “pharmaceutically acceptable carrier” includes vehicles and diluents.

The formulations include those suitable for oral, parenteral (including subcutaneous, intradermal, intramuscular, intravenous and intraarticular), rectal administration, as well as those for administration by inhalation. The most suitable route may depend upon the condition of the recipient and the purpose of the MR imaging experiment. The formulations may conveniently be presented in unit dosage form and may be prepared by any of the methods well known in the art of pharmacy. All methods include the step of bringing into association a compound of the invention (“active ingredient”) with the diluent which constitutes one or more accessory ingredients. In general, the formulations are prepared by uniformly and intimately bringing into association the active ingredient with liquid carriers or finely divided solid carriers or both and then, if necessary, shaping the product into the desired formulation. Generally accepted formulations are well known in the art. See, for example, Remington: The Science and Practice of Pharmacy, A. R. Gennaro, ed. (1995), the entire disclosure of which is incorporated herein by reference.

Pharmaceutical compositions containing one or more redox active probes may be conveniently presented in unit dosage form and prepared by any of the methods well known in the art of pharmacy. Exemplary unit dosage formulations are those containing an effective dose, or an appropriate fraction thereof, of the active ingredient, or a pharmaceutically acceptable salt thereof. The magnitude of a diagnostic dose typically varies with the nature of the redox system to be queried and the route of administration. The dose in the formulation, and perhaps the dose frequency, will also vary according to the age, body weight and response of the individual patient. In general, the total dose (in single or divided doses) ranges from about 1 mg to about 7000 mg, preferably about 1 mg to about 100 mg, and more preferably, from about 10 mg to about 100 mg, and even more preferably from about 20 mg to about 100 mg.

In various embodiments, the invention provides formulations in which the hyperpolarized MR imaging agent is present in a concentration of about 10 mM to about 10M, e.g., about 50 mM to about 500 mM. For various embodiments in which bolus injection is utilized, the concentration is from about 0.1 mM to about 10M, e.g., from about 0.2 mM to about 10M, e.g., from about 0.5 mM to about 1M, e.g., from about 1.0 mM to about 500 mM, e.g., 10 mM to 300 mM.

It is further recommended that children, patients over 65 years old, and those with impaired renal or hepatic function, initially receive low doses and that the dosage is titrated based on individual responses and/or blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

It should be understood that in addition to the ingredients particularly mentioned above, the formulations of this invention may include other agents conventional in the art having regard to the type of formulation in question, for example those suitable for oral administration may include flavoring agents.

Formulations of the present inventions suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. The pill and capsule formulations are convenient vehicles for preparation and storage of a unit dosage formulation. When the compound is in a solid form in the pharmaceutical formulation, it is generally dissolved in a pharmaceutically acceptable diluent prior to polarization and use.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described. Formulations for rectal administration may be presented as a suppository with the usual carriers such as cocoa butter or polyethylene glycol. Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

The pharmaceutically acceptable diluent may take a wide variety of forms, depending on the route desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets. Exemplary solid oral preparations are tablets or capsules, because of their ease of administration. If desired, tablets may be coated by standard aqueous or nonaqueous techniques. Oral and parenteral sustained release dosage forms may also be used.

In an exemplary embodiment, the pharmaceutical formulation is a unit dosage formulation. In this embodiment, the compound of the invention is present in an amount needed to administer sufficient compound to a subject to perform a desired imaging experiment, or series of imaging experiments. In an exemplary unit dosage formulation, a compound of the invention is present in an amount sufficient to provide a dosage of from about 0.5 mg/kg to about 50 mg/kg, e.g., from about 1 mg/kg to about 25 mg/kg. In an exemplary embodiment, the compound of the invention is present in the dosage formulation in an amount sufficient to provide a dosage of from about 0.5 mg/kg to about 5 mg/kg, e.g., from about 0.7 mg/kg, e.g., about 1 mg/kg.

For in vivo use, a hyperpolarized solid MR imaging agent is preferably dissolved in a liquid administrable media (e.g., water or saline), administered to a subject and an MR image recorded. Thus solid MR imaging agents are preferably rapidly soluble (e.g., water soluble) to assist in formulating administrable media. In an exemplary embodiment, the MR imaging agent dissolves in a physiologically tolerable carrier (e.g., water or saline) to a concentration of at least 1 mM. In an exemplary embodiment, the compound of the invention is hyperpolarized in the solid state by, for example, a dynamic nuclear polarization technique, and the hyperpolarized compound is subsequently formulated for administration by the desired route.

In various embodiments, the hyperpolarized compound is initially dissolved at a concentration higher than the concentration at which it will be administered. The concentrated hyperpolarized compound is diluted to a concentration appropriate for administering to a subject. In various embodiments, the compound is dissolved at an administrable concentration and it is this solution that is hyperpolarized.

In an exemplary embodiment, the compound of the invention is formulated with another agent. Exemplary agents include other MR sensitive compounds (e.g., paramagnets, other hyperpolarized compounds) and other diagnostic agents. In various embodiments, the compound of the invention is formulated with an agent that alters the behavior of the redox system being queried by the compound of the invention. These compounds include therapeutic and diagnostic agents.

Methods

The present invention provides in vivo and in vitro methods for querying a redox system using MR in combination with a hyperpolarized compound of the invention. The method includes administering a hyperpolarized compound of the invention to a subject (or system) and recording an MR image of a region of the subject including the hyperpolarized agent of the invention.

In various embodiments, the invention provides methods for imaging and characterizing redox relevant processes in vivo. The method includes administering to a subject a detectable amount of a hyperpolarized probe of the invention an acquiring an MR image post-administration. The MR image includes a region in which the hyperpolarized agent is distributed. In an exemplary embodiment, this new MRI technique to probe specific enzymatic pathways utilizes a ¹³C-labeled substrate with a long spin-lattice relaxation time (T₁), whose metabolic product is readily distinguishable by chemical shift. In an exemplary embodiment, this technique is utilized to yield dynamic modeling of rapid enzymatic fluxes in vivo, (see, e.g., the rapid conversion of [1-¹³C]pyruvate to [1-¹³C] lactate as mediated by lactate dehydrogenase (LDH)).

In an exemplary embodiment, the invention provides an in vivo method to probe the dynamic reducing capacity of tissues via GSH-mediated conversion of the probe to its redox partner. In an exemplary embodiment [1-¹³C] DHA is used to probe the dynamic reducing capacity of tissues in vivo, via GSH-mediated conversion to Vitamin C.

In an exemplary embodiment, the method of the invention is carried out within the time that the MR imaging agent remains significantly polarized. Thus, in various embodiments, once nuclear spin polarization and dissolution has occurred, the administration of the MR imaging agent is effected rapidly and the MR measurement follows shortly thereafter. Accordingly, it is generally preferred that the subject or system is available and close to the area in which the polarization has been carried out. If this is not possible, the polarized compound of the invention should be transported to the relevant area, preferably at low temperature.

An exemplary administration route for the polarized MR imaging agent is parenteral e.g., by bolus injection, by intravenous, intraarterial or peroral injection. The injection time should be scaled to the T₁ of the hyperpolarized compound. For example, an injection time equivalent to 5 T₁ or less, 3 T₁ or less, T₁ or less, or even 0.1 T₁ or less is generally preferred.

The lungs and other components of the airway may be imaged by spray, e.g. by aerosol spray.

For use in in vivo imaging, the formulation, which preferably will be substantially isotonic, may conveniently be administered at a concentration sufficient to yield a concentration of the MR imaging agent in the imaging zone of from about 1 μM to about 1M; however, the precise concentration and dosage will of course depend upon a range of factors such as toxicity, the organ targeting ability of the MR imaging agent, and the administration route. The optimum concentration for the MR imaging agent represents a balance between-various factors. In exemplary embodiments, optimum concentrations lie in the range about 0.1 mM to about 10M, e.g., about 0.2 mM to about 1M, e.g., about 0.5 to about 500 mM.

Formulations of use in the methods of the invention for intravenous or intraarterial administration preferably contain the MR imaging agent in concentrations of 10 mM to 10M, especially 50 mM to 500 mM. For bolus injection the concentration may conveniently be 0.1 mM to 10M, preferably 0.2 mM to 10M, more preferably 0.5 mM to 1M, still more preferably 1.0 mM to 500 mM, yet still more preferably 10 mM to 300 mM.

The dosages of the MR imaging agent used in the method of the present invention will vary according to the precise nature of the MR imaging agents used, of the tissue or organ of interest and of the measuring apparatus. Preferably the dosage should be kept as low as possible whilst still achieving a detectable contrast effect. In various embodiments, the dosage is approximately 10% of LD₅₀, e.g., in the range Ito 1000 mg/kg, e.g., 2 to 500 mg/kg, e.g., 3 to 300 mg/kg. As discussed above, these amounts can be delivered in single (i.e., unit) or multiple dosages.

In another embodiment, the invention provides a means of functional MR imaging of a component of a redox system. In an exemplary embodiment, the functional imaging detects the activity of a receptor implicated in the redox system.

In various embodiments, the invention provides systems and methods for determining the amount of a probe of the invention that has been taken up a by a cell or tissue. In an exemplary embodiment, this amount is determined by detecting an NMR signal corresponding to a probe of the invention which has been reduced or oxidized by the intracellular conditions (and/or intercellular conditions in a tissue) and evaluating the signal. The evaluation can be qualitative or quantitative. In various embodiments, this method is utilized to detect active transport of the probe into a cell. In an exemplary embodiment, the transports the probe into a cell. In an exemplary embodiment, the receptor is a glucose receptor.

In further exemplary embodiments, the invention provides systems and in vitro methods of probing redox systems in organ systems, perfused cell systems and in enzyme systems. These methods find use in elucidating the mechanism of redox activity in a particular system and in drug discovery and optimization. The method consists of administering to the system a hyperpolarized compound of the invention and acquiring an MR image of a region of the system containing the hyperpolarized compound of the invention. Exemplary systems of use in the present invention include those known in the art, such as those discussed in Keshari et al. Hyperpolarized (13)C spectroscopy and an NMR-compatible bioreactor system for the investigation of real-time cellular metabolism. Magnetic Resonance in Medicine: Official Journal of the Society of Magnetic Resonance in Medicine/Society of Magnetic Resonance in Medicine (2010) vol. 63 (2) pp. 322-329.

In another embodiment, the invention provides systems and methods for querying the redox state, redox potential and other redox-relevant properties in a biological or non-biological system. For example, the redox conditions within a reaction mixture containing a reducing agent, an oxidizing agent or a combination of the two can be probed by measuring the ¹³C NMR of the hyperpolarized probe of the invention in the reaction mixture. See, for example, FIG. 1d.

The headings and subheadings utilized herein are not intended to limit the scope of the present inventions.

The following examples are provided to illustrate selected embodiments of the present inventions and are not to be construed as limiting its scope.

### EXAMPLES

### Example 1

### Materials and Methods

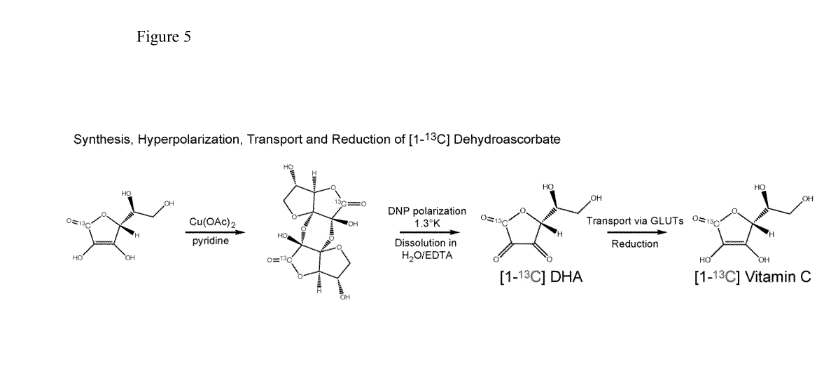

Hyperpolarized [1-¹³C]-DHA and [1-¹³C]-vitamin C: [1-¹³C] DHA (Isotec, Miamisburg, Ohio) was synthesized using a published method (FIG. 5) A 2.2M solution of [1-¹³C] DHA in dimethyacetamide (DMA) containing 15 mM OX063 trityl radical (Oxford Instruments) was hyperpolarized on a HyperSense DNP instrument (Oxford Instruments) as previously described. The frozen sample was dissolved in distilled water containing 0.3 mM ethylenediaminetetracetic acid (EDTA). Similarly, a 2.2M solution of [1-¹³C] Vitamin C (Omicron, South Bend, Ind.) was prepared as a sodium salt in NaOH/water/dimethyl sulfoxide (DMSO) containing 15 mM OX063. This compound was polarized by an identical method and dissolved in 100 mM phosphate buffer, pH 7.0.

### 11.7 T NMR Studies:

NMR studies were performed on an 11.7 T Varian NOVA spectrometer (125 MHz ¹³C, Varian Instruments) using a 10 mm ¹⁵N/³¹P/¹³C broadband direct detect probe. Thermal and dynamic hyperpolarized spectra for [1-¹³C] DHA and [1-¹³C] Vitamin C were obtained as previously described, and used to calculate T₁'s and signal enhancements. For NMR studies of the reduction of [1-¹³C] DHA to [1-¹³C] Vitamin C, hyperpolarized [1-¹³C] DHA was reacted with an excess of sodium cyanoborohydride (NaBH₃CN).

### 3 T Studies:

T₁ studies were performed using a 3 T MRI scanner (GE Healthcare, Waukesha, Wis.) equipped with the MNS (multinuclear spectroscopy) hardware package. The RF coil used in these experiments was a dual-tuned ¹H-¹³C coil with a quadrature ¹³C channel and linear ¹H channel construction used in ¹³C mouse imaging studies. ¹³C MRSI studies were carried out as previously published. 350 μl (mice) or 2 mL (rats) of a hyperpolarized 15 mM [1-¹³C] DHA solution were injected similar to previously described methods for [1-¹³C]pyruvate. Similar experiments were performed on a set of normal mice using hyperpolarized [1-¹³C] Vitamin C at 50 mM.

### Results:

### Synthesis, Polarization, and In Vitro Validation of [1-¹³C] DHA

In vitro methods to interrogate redox chemistry have relied on designer redox-sensitive fluorescent proteins (yellow- or green fluorescent protein) incorporating an artificial dithiol-disulfide pair, or on detection of ROS themselves via chemical switching. In general, fluorescent probes have limited clinical translation given poor tissue penetration; making MR-compatible hyperpolarized ¹³C probes an attractive alternative. The technique is generally limited to endogenous biomolecules, given the relatively high concentrations of metabolic probes injected. Since the hyperpolarized ¹³C signal can only be observed 1-2 minutes in vivo, rapid uptake and metabolism is an additional important requirement. The redox probe [1-¹³C] DHA was designed with these general requirements in mind, minimizing T₁-dependent signal loss by dipolar coupling, and having sufficient chemical shift separation from [1-¹³C] Vitamin C for in vivo MRSI. [1-¹³C] DHA was prepared as a pure dimer by air oxidation of [1-¹³C] ascorbic acid in the presence of catalytic amounts of copper (II) acetate, according to a published procedure (FIG. 5). The material was polarized by using a DNP approach and dissolved with a hot water/EDTA solution, with T₁'s calculated as previously described. T₁'s for the C_{1 [}1-¹³C] DHA and [1-¹³C] Vitamin C carbons are tabulated at both 11.7 T and 3 T, with the longest recorded T₁ that of [1-¹³C] DHA at 3 T, 57 s (FIG. 1c, Table 1). There is a significant increase in T₁ relaxation of the carbon of interest with decreasing field strength. Due to chemical shift anisotropy (CSA), carbonyl carbons tend to decrease in T₁, leading to faster polarization decay with higher field strengths. The polarizations for [1-¹³C] DHA and [1-¹³C] Vitamin C were measured as 5.9±0.5% and 3.6±0.1% respectively, representing signal enhancements on the order of approximately 24,000 and 15,000-fold relative to thermal equilibrium at 3 T.

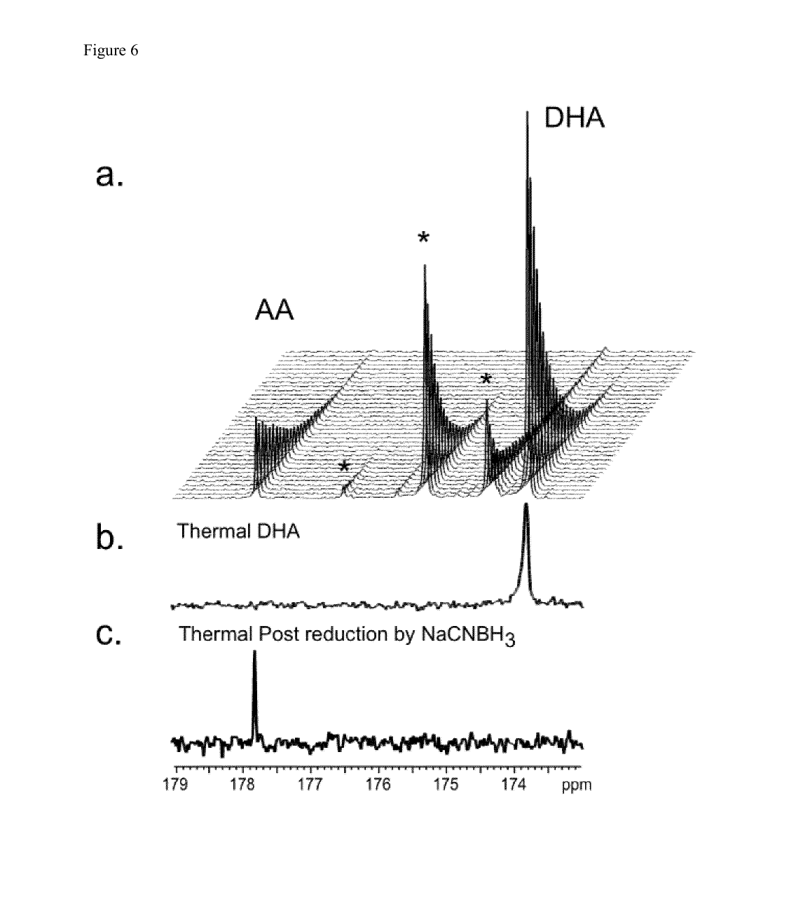

The chemical shift separation of the anticipated metabolite [1-¹³C] Vitamin C from the parent DHA compound was confirmed by chemical reduction in vitro analogous to the GSH—mediated conversion in cells. The reaction of hyperpolarized [1-¹³C] DHA with NaBH₃CN yields the reduced [1-¹³C] Vitamin C within seconds, as described in FIG. 1d. Due to the higher stability of the DHA lactone at lower pH, these experiments were conducted in unbuffered solution at 37° C. The equilibrium NMR spectrum of the purified dimeric [1-¹³C] DHA demonstrated a single peak at 174 ppm. Following polarization, dissolution, and reaction with NaBH₃CN a more complex spectrum was obtained, with evidence of rapid formation of [1-¹³C] Vitamin C. On increasing concentrations of NaBH₃CN, enhanced rates of conversion to [1-¹³C] Vitamin C were observed indicating dependence on the reducing potential of the solution. Fully relaxed thermal data acquired immediately following these studies demonstrated only a single peak corresponding to [1-¹³C] Vitamin C, confirmed by chemical shift, indicating that the reaction had gone to completion (FIG. 6).

### Dynamic Magnetic Resonance Spectroscopy Confirms Rapid Reduction of Hyperpolarized [1-¹³C] DHA In Vivo

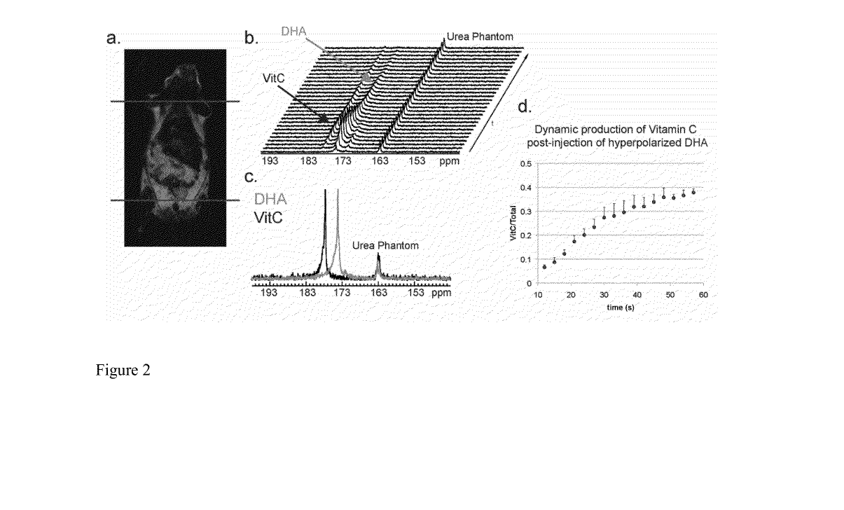

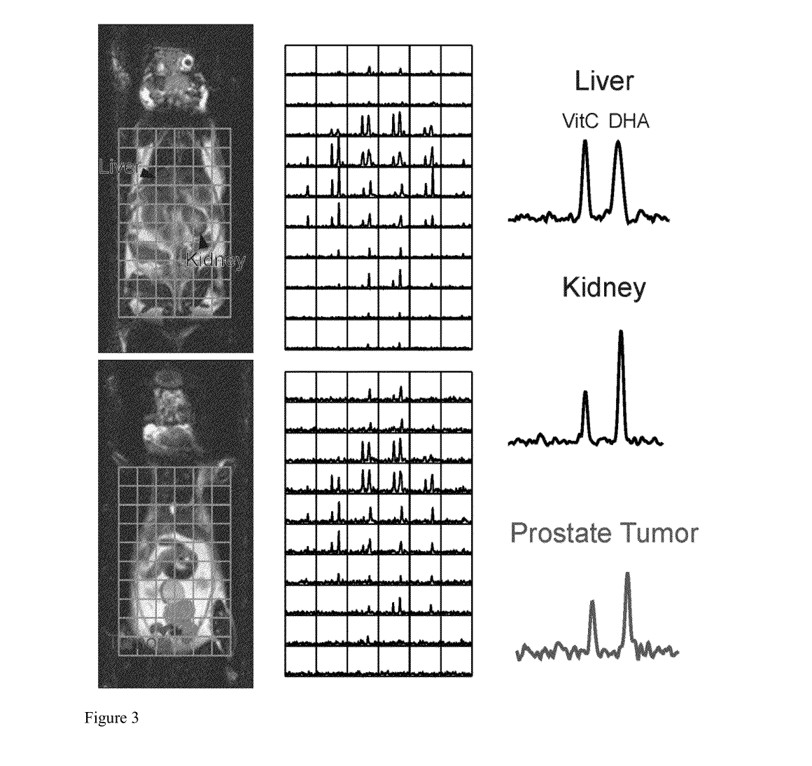

Evidence of in vivo transformation was determining having obtained data validating hyperpolarized [1-¹³C] DHA as a redox probe. The transport of DHA in vivo occurs by a mechanism analogous to that of glucose, by facilitated diffusion using GLUT1, GLUT3, and GLUT4. Since glucose is known to compete with the uptake of DHA, in vivo experiments were performed on animals (mice and rats) fasted overnight. Animals were also pre-treated with a DHA dose identical to that administered during the imaging experiment, 1 hour prior which has been shown to reduce the physiologic effects of DHA administration. Dynamic MRSI studies were carried out as previously published. 350 μl of a hyperpolarized 15 mM [1-¹³C] DHA solution were injected similar to previously described methods for [1-¹³C] pyruvate, in normal mice with a ¹³C urea phantom present for chemical shift reference. In a separate set of normal mice, similar experiments were performed using hyperpolarized [1-¹³C] Vitamin C at a slightly higher concentration, 50 mM. FIG. 2 demonstrates the metabolism following injection of 15 mM hyperpolarized [1-¹³C] DHA in a normal mouse. The DHA resonance at 174 ppm reached a maximum at 19±1.7 secs post injection. A large metabolite resonance was observed at 177.8 ppm, reaching a maximum at 29±1.7 sec, consistent with rapid conversion to [1-¹³C] Vitamin C in vivo. This chemical shift was confirmed both in vitro at neutral pH using ¹³C urea as a reference, as well as in vivo with hyperpolarized [1-¹³C] Vitamin C itself injected in a separate animal. For the latter experiment, trace oxidation to [1-¹³C] DHA was observed in vivo (Supplementary FIG. 3). These results are concordant with the literature regarding the bioconversion and uptake of DHA, reflected in the relative steady-state concentrations of DHA and Vitamin C in cells.

### Reduction of Hyperpolarized [1-¹³C] DHA In Vivo Localizes to Kidneys, Liver, Brain, and Tumor

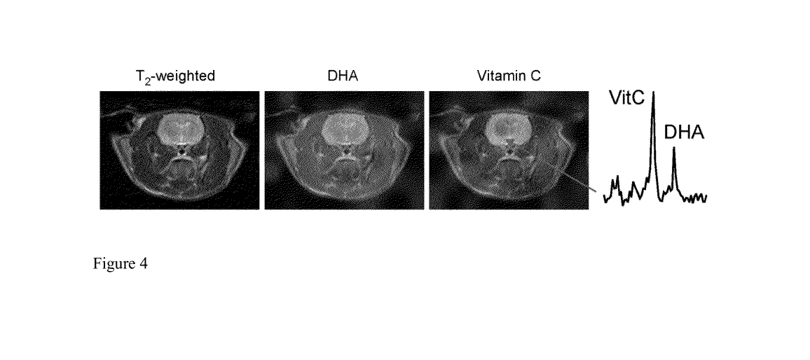

Tissues with high exposure to environmental toxins, namely the liver, kidney and lungs, are known to be rich in GSH and were considered the most likely source for the hyperpolarized [1-¹³C] Vitamin C signal observed in vivo. In addition, a contribution from highly glycolytic tissues was anticipated, given the rapid transport properties of dehydroascorbate. Analogous to experiments performed previously using hyperpolarized [1-¹³C]pyruvate, 3D MRSI studies were conducted on a series of normal mice (n=3), TRAMP mice (n=3), and rats (n=3) using a dual-tuned RF coil. Rapid conversion to Vitamin C was observed in the murine liver (VitC/Total Carbon of 0.41±0.05, n=25 voxels) and kidney (VitC/Total Carbon of 0.30±0.05, n=16 voxels) (FIG. 3). In tumors of TRAMP mice VitC/Total Carbon was 0.29±0.09 (n=9 voxels), while no Vitamin C signal was observed in the prostate region of normal mice. MRSI data acquired from rat brains demonstrated an even greater ratio of VitC/Total Carbon of 0.51±0.1 (n=16 voxels) with Vitamin C localized to the brain while DHA was present in the surrounding muscle tissue (FIG. 4).

### Discussion:

Alterations in cellular redox are implicated in a large number of pathologic processes, with a growing body of evidence also suggesting that controlled ROS chemistry is essential for normal development (28). Given the universality of redox chemistry in the functional cell, hyperpolarized [1-¹³C] DHA was developed, which is an MR-compatible endogenous probe to interrogate populations of key redox pairs in vivo, in particular GSH/GSSG which are coupled to intracellular DHA reduction. The principal advantage of hyperpolarized ¹³C MRI over other molecular imaging modalities is detection of ¹³C metabolites by chemical shift, allowing real-time evaluation of physiologically relevant enzyme fluxes in vivo. Data obtained are potentially confounded by differential perfusion of (and transport into) tissues of interest, a problem that has been addressed by co-polarization of ¹³C probes with a perfusion agent, for example the intravascular agent ¹³C urea. The in vivo conversion of hyperpolarized [1-¹³C] DHA is related both to its rapid transport into highly glycolytic cells, and the reducing environment within those tissues mediated most directly by GSH. Hyperpolarized [1-¹³C] DHA was reduced rapidly to Vitamin C within the liver, as might be expected given both its high perfusion and role in GSH-dependent detoxification of electrophilic substances. To a lesser extent, the [1-¹³C] Vitamin C metabolite was observed within kidneys and TRAMP tumors, the latter demonstrating both elevated glycolysis and high concentrations of GSH.

The concentration of Vitamin C in the brain is remarkably high, estimated to be 10 mM in neurons, while glia harbor high concentrations of GSH. This high ascorbate level is generally attributed to the high rate of oxidative metabolism in neurons, making the brain particularly vulnerable to ROS and ischemia/reperfusion injury. Total brain Vitamin C levels are under strong homeostatic regulation, with extracellular Vitamin C concentrations mediated in part by heteroexchange with glutamate. High levels of brain Vitamin C are neuroprotective, and may be enhanced by administration of DHA, which readily crosses the blood-brain barrier. On injection of hyperpolarized [1-¹³C] DHA into a normal rat, significant reduction to [1-¹³C] Vitamin C was observed within the brain, with essentially no background conversion observed in surrounding tissues. Given the limited anatomic resolution of this study, we were not able to determine whether this reduction took place primarily in gray matter (dominated by neurons) or white matter (predominantly glia). Rapid reduction of hyperpolarized [1-¹³C] DHA within the brain suggests a role for this probe in functional imaging (GLUT transporter density), determining local glutamate concentrations, as well as predicting vulnerability to ROS.

Table 1 shows chemical shifts, T₁ spin-lattice relaxation constants, solid-state build-up constants and percent polarization for ascorbates. Chemical shifts are relative to ¹³C-urea at 163 ppm. All measurements are reported as mean±standard deviation.

TABLE 1

| | | | | Buildup | Percent |
|---|---|---|---|---|---|
| | | | 11.7T T₁ | Constant | Polariza- |
| | δ (ppm) | 3T T₁ (s) | (s) | (s) | tion |
| [1-¹³C] DHA | 174.0 | 56.5 ± 7.6 | 20.5 ± 0.9 | 1120 ± 50 | 5.9 ± 0.5 |
| [1-¹³C] | 177.8 | 29.2 ± 2.5 | 16.0 ± 0.8 | 1756 ± 97 | 3.6 ± 0.1 |
| Vitamin C | | | | | |

### Example 2

### Hyperpolarized ¹³C Ascorbates in the Anesthetized Rat Brain

### Introduction

Reduction and oxidation (redox) chemistry is involved in both normal and abnormal brain function, in processes as diverse as circadian rhythms and neurotransmission. Intracellular redox is maintained by coupled reactions involving NADPH, glutathione (GSH), and vitamin C (VitC), as well as their corresponding oxidized counterparts. The reducing agents GSH and VitC are maintained at high concentrations in the brain, and have a critical role in dealing with reactive oxygen species (ROS) seen as culprits in aging, neurodegenerative disease, and ischemic injury. We have developed [1-¹³C] dehydroascorbate [DHA], the oxidized form of VitC, as an endogenous redox sensor for in vivo imaging using hyperpolarized (HP) ¹³C spectroscopy. In contrast to VitC, DHA readily crosses the blood-brain-barrier (BBB) and may play a key role in maintaining cerebral ascorbate levels. The goal of this study was to compare HP [1-¹³C] DHA and HP [1-¹³C] VitC in the normal brain.

### Methods

Synthesis of [1-¹³C] DHA: [1-¹³C] DHA (Isotec, Miamisburg, Ohio) was synthesized as in Example 1. Hyperpolarization and dissolution of [1-¹³C] DHA and [1-¹³C] Vitamin C: A 2.2M solution of [1-¹³C] DHA in dimethyacetamide (DMA) containing 15 mM OX063 trityl radical (Oxford Instruments) was hyperpolarized on a HyperSense DNP instrument (Oxford Instruments). Similarly, a 2.2M solution of [1-¹³C] Vitamin C (Omicron, South Bend, Ind.) was prepared as a sodium salt in NaOH/water/dimethyl sulfoxide (DMSO) containing 15 mM OX063. This compound was polarized by an identical method and dissolved in 100 mM phosphate buffer, pH 7.0. 3 T Studies: In vivo 2D ¹³C MRSI studies were performed using a 3 T MRI scanner (GE Healthcare, Waukesha, Wis.) equipped with the MNS (multinuclear spectroscopy) hardware package with 5 mm×5 mm in-plane resolution (slab thickness 20 mm) 2 mL of 15 mM HP [1-¹³C] DHA solution were injected similar to previously described methods for [1-¹³C]pyruvate. Similar experiments were performed using HP [1-¹³C] Vitamin C at 50 mM. In all cases the imaging sequence was initiated 10 s following completion of a 15 s injection (representing a total delay of 25 s). Data Processing and Analysis: MRSI data was processed using custom software written in IDL 8 (ITT Visual Information Solutions, CO, USA) and Matlab 2009b (MathWorks, MA, USA). DHA and Vitamin C resonances were integrated and peak heights were used to calculate relevant ratios. Average metabolite ratios (VitC/[VitC+DHA]) were calculated for voxels corresponding to brain and surrounding tissues.

### Results

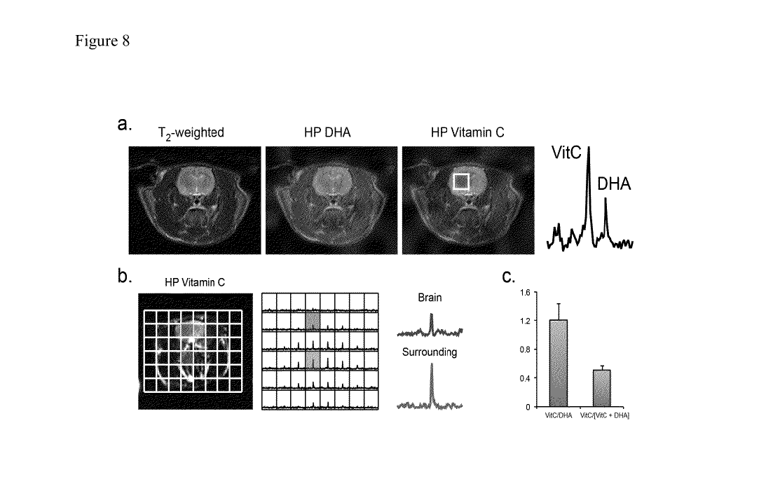

The transport of DHA in vivo occurs by a mechanism analogous to that of glucose, by facilitated diffusion using GLUT1, GLUT3, and GLUT4. Since glucose is known to compete with the uptake of DHA, in vivo experiments were performed on rats fasted overnight. Animals were also pre-treated with a DHA dose identical to that administered during the imaging experiment, 1-hour prior, which has been shown to reduce the physiologic effects of DHA administration. Data obtained are summarized in FIG. 8. For HP [1-¹³C] DHA studies (FIG. 8a), voxels corresponding to brain were compared with those within in the surrounding soft tissues. For brain voxels (n=16) the average VitC/[VitC+DHA] was 0.51±0.10 and average VitC/DHA was 0.51±0.10 (FIG. 8c). Remarkably, no VitC resonances were observed in voxels outside the brain. In contrast, following injection of HP [1-13C] VitC, no observable oxidation to HP [1-¹³C] DHA (or other metabolite) was observed. In these studies, the magnitude of the [1-¹³C] VitC resonance was higher in surrounding tissues than in the brain (FIG. 8b).

### Discussion

The steady-state concentration of Vitamin C in the brain is remarkably high, estimated to be 10 mM in neurons, while glia harbor high concentrations of GSH. This high Vitamin C level is generally attributed to the high rate of oxidative metabolism in neurons, making the brain particularly vulnerable to ROS and ischemia/reperfusion injury. Total brain Vitamin C levels are under strong homeostatic regulation, with extracellular Vitamin C concentrations mediated in part by heteroexchange with glutamate. High levels of brain Vitamin C are neuroprotective, and may be enhanced by administration of DHA, which readily crosses the blood-brain barrier. On injection of HP [1-¹³C] DHA into a normal rat, significant reduction to [1-¹³C] Vitamin C was observed within the brain, with no background conversion observed in surrounding tissues. Given the limited spatial resolution of this study, we were not able to determine whether this reduction took place primarily in gray matter (dominated by neurons) or white matter (predominantly glia). Rapid reduction of HP [1-¹³C] DHA within the brain suggests a role for this probe in functional imaging (GLUT transporter density), determining local glutamate concentrations, as well as predicting vulnerability to ROS.

### Example 3

### Molecular Imaging of Non Alcoholic Fatty Liver Disease Using an Endogenous Hyperpolarized Redox Sensor

### Introduction

Non-alcoholic fatty liver disease (NAFLD) is recognized as the most prevalent liver abnormality in the United States, with nearly 10% of the population demonstrating some form of the disease. Incidence can reach as high as 70% in patients who are obese and/or have type II diabetes. Many rodent models have been developed to study NAFLD, which are induced by both diet and genetic manipulation. The methionine choline deficient (MCD) model has been used to develop NAFLD, with onset of disease readily visible after 2 weeks on the diet. Changes in reduction and oxidation (redox) have been implicated in the development of this disease as well as its response to therapy. Methods to detect redox changes in these animal models non-invasively are limited. Recent development of hyperpolarized (HP) [1-¹³C] dehydroascorbate (DHA), using the dissolution dynamic nuclear polarization (DNP) technique, provides a new redox sensor to address NAFLD and its treatment. HP DHA is readily transported into the cell and reduced to Vitamin C (VitC) in vivo. The aim of this study was to use HP [1-¹³C] DHA to image redox changes in a standard model of NAFLD and correlate these findings with standard 1H imaging and histopathology.

### Methods

Prior to and post diet, high-resolution T2 and fat-water imaging was conducted at 14 T using a Varian WB600 micro-imager and 40 mm 1H millipede coil (Varian Instruments, Palo Alto, Calif.). Lipid percentage maps were calculated from fat and water images acquired using a conventional spin-echo sequence using the Dixon method. 350 μL of hyperpolarized DHA was injected each mouse (n=4) at baseline and 2 weeks of MCD diet using a Hypersense (Oxford Instruments). 13C MRSI data was acquired using an EPSI readout, variable flip angle scheme, matrix size 16×8×8 and final voxel resolution of 6 mm isotropic on a 3 T GE MRI (GE Healthcare, Waukesha, Wis.) equipped with a multinuclear package and dual-tuned 1H-¹³C imaging coil. MR data was processed offline using custom software written in IDL 8 (ITT Visual Information Solutions, CO, USA) and Matlab 2009b (MathWorks, MA, USA). Ratios of HP DHA and VitC were calculated from the peak integrals in 3D MRSI data. Mice were then sacrificed and liver tissue was sectioned for staining with hematoxylin and eosin (H&E, structure) and Oil Red (lipid presence). Livers of normal mice were used for histologic comparison.

### Results and Discussion

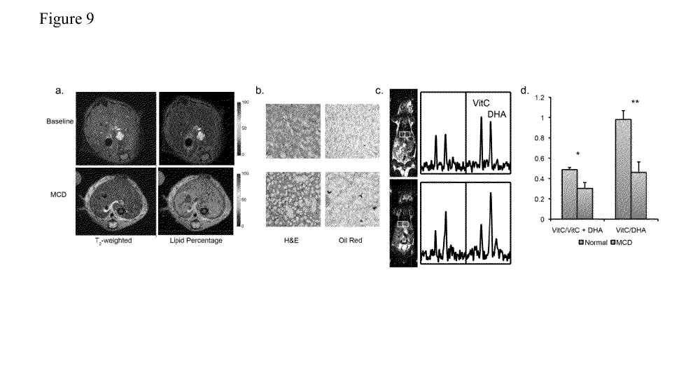

The normal liver demonstrates high conversion of HP DHA to Vitamin C (VitC/VitC+DHA of 0.49±02), indicative of high antioxidant capacity. We believe that this antioxidant capacity correlates most strongly with the concentration of reduced glutathione (GSH), which is high in the normal liver (on the order of 5 mM) but decreased significantly in experimental fatty liver models. When placed on the MCD diet for two weeks, the average mouse weight decreases 13% and the liver fat percentage dramatically increases 6 fold from baseline (FIG. 9a). These changes are validated in histopatholgic sections of the same liver tissue (FIG. 9b) as compared to those of normal. Lipid staining denotes large regions of fat accumulation in the liver (FIG. 9b, Oil Red staining), which is also visualized by fat/water imaging at high field (FIG. 9a), while the liver size does not change significantly. There is also a remarkable decrease in HP VitC after 2 weeks on the diet (FIG. 9c) resulting in a 48% decrease in the VitC/VitC+DHA ratio from baseline (0.49±02 to 0.30±0.5, P=0.01) and 92% in the VitC/DHA ratio (0.98±0.09 to 0.46±0.1, P=0.005). Deregulation of lipid export and changes in oxidative stress are hallmarks of fatty liver disease, with overload of free fatty acids resulting in electron leakage during mitochondrial β-oxidation. Generation of lipid peroxides results in subsequent damage to hepatic membranes, proteins and DNA. Total anti-oxidant capacity, both enzymatic and non-enzymatic is insufficient to mitigate liver injury. After 2 weeks of the MCD diet, increased oxidative stress leads to depletion in hepatic reducing capacity, thus resulting in lowered production of HP VitC. The non-invasive observation of changes in redox provides a means to evaluate not only the extent of this liver injury but also potential response to antioxidant therapeutics.

### CONCLUSIONS

This study demonstrates the first application of HP MR to a non-oncogenic liver pathology, namely NAFLD that impacts 1 out of 10 Americans in their lifetime. Redox has been implicated in the progression of this disease and remains an active target for NAFLD treatment. HP DHA conversion to Vitamin C provides a potential non-invasive probe for changes in redox. In this study, we demonstrate changes in redox with onset of NAFLD in a MCD deficient mouse model. Future studies aim to extend the use of HP DHA for fatty liver disease characterization in other models as well as response to both therapeutics and diet.

### Example 4

### Comparison of Hyperpolarized [1-¹³C] Dehydroascorbate-MR and FDG-PET in a Transgenic Prostate Cancer Model

### Introduction

Hyperpolarized (HP) 13C MR studies of the transgenic model of prostate cancer (TRAMP) model have demonstrated that hyperpolarized (HP) [1-¹³C]pyruvate can detect both local and metastatic prostate cancer, provide an assessment of pathologic grade, and determine early response to therapy. This finding has lead to the recent clinical translation of HP [1-¹³C]pyruvate in the first human trial of HP MR in prostate cancer patients. Recently, we have developed a new probe for in vivo imaging of reduction and oxidation (redox), HP [1-¹³C] dehydroascorbate (DHA). HP DHA is rapidly taken into the cell via glucose (GLUT) transporters and then reduced to ascorbate (VitC). This conversion takes place rapidly in the brain, liver, kidneys, and TRAMP tumor. These tissues are known to harbor large concentrations of glutathione (GSH), primarily responsible for reduction of DHA in vivo in coupled redox reactions with NADPH, mediated by several intracellular enzymes including glutaredoxin. Our hypothesis is that the observed reduction of HP [1-¹³C] DHA in vivo relates to both cellular GSH concentration, as well as increased glucose uptake. The gold standard for metabolic imaging, 2-18F-2-deoxy-D-glucose (FDG) used in positron emission tomography (PET) imaging, takes advantage of increased glucose uptake to generate contrast between tumor and normal tissues. Since [1-¹³C] DHA has an essentially identical transport (and charge trapping) mechanism, we speculated that it might have considerable overlap with FDG in tumor models. The goal of this study was to compare the increased HP [1-¹³C] VitC signals observed by MRSI to accumulation of FDG-PET in a cohort of TRAMP mice.

### Methods

Hyperpolarization and dissolution of [1-¹³C] DHA: [1-¹³C] DHA dimer was polarized and dissolved. 3 T MR Studies: 3D MRSI studies were performed using a 3 T MRI scanner (GE Healthcare, Waukesha, Wis.) equipped with the MNS (multinuclear spectroscopy) hardware package and a dual-tuned mouse coil as previously described. Data Processing and Analysis: In vivo MRSI data was processed using custom software written in IDL 8 (ITT Visual Information Solutions, CO, USA) and Matlab 2009b (MathWorks, MA, USA). Average metabolite ratios (VitC/[VitC+DHA]) were calculated for voxels corresponding to both tumor and surrounding benign tissues in TRAMP mice (N=4). FDG-PET: PET scans were performed on a small animal PET/CT scanner (Inveon, Siemens Healthcare, Malvern, Pa.). Mice were fasted overnight, anesthetized, and injected intravenously with 150-170 uCi FDG in 0.1-0.2 mL saline. PET images were acquired 50 minutes post injection in one 600 second frame. CT images were acquired in 120 projections of continuous rotation to cover 220° with x-ray tube operating at 80 kVp, 0.5 mA, and 175 ms exposure time. The matrix size of the reconstructed CT images was 512×512×662 with an isotropic voxel size of 0.191×0.191×0.191 mm³ PET images were reconstructed using a manufacturer-provided ordered subsets expectation maximization (OS-EM) algorithm resulting in a 128×128×159 matrices with a voxel size of 0.776×0.776×0.796 mm³ ROIs were drawn in both the tumors and surrounding tissue to calculate relative tumor signals.

### Results and Discussion

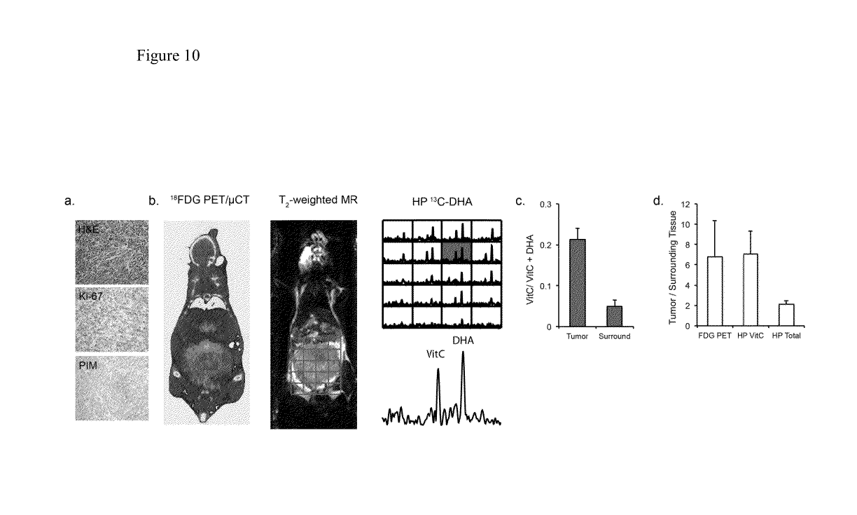

Relative to benign prostate tissue, TRAMP tumors (N=4) demonstrated increased cellularity (100% poorly differentiated), proliferative rates (88±7% KI-67 positive cells) and hypoxia (19.4±6% PIM positive cells) (FIG. 10a). These cells tend to be highly glucose avid and reduced. High levels of HP VitC are observed in these TRAMP tumors post-injection of HP DHA (0.23±0.03) significantly higher than that of surrounding tissues (0.05±0.02, P=0.005, FIG. 10c) and benign prostate (0.06±0.03, P=0.001). When imaged using PET-FDG these mice also demonstrated increased ¹⁸F signal in the regions of tumor relative to surrounding regions (6.8±3.6, FIG. 10d). ¹⁸FDG signals in the normal prostate are difficult to quantify because of its proximity to the bladder. ¹⁸FDG signals in the TRAMP tumors appeared fairly homogenous (FIG. 10b). Total HP¹³C in tumors was elevated relative to surrounding tissue (2.1±0.4), but this was significantly less than the tumor HP VitC/[VitC+DHA] relative to surrounding tissue (7.0±2.3). Interestingly, regions of differing VitC/[VitC+DHA] were seen in the tumors that may represent different concentrations of reducing agents. Future studies will be needed to elucidate these regional differences in reducing capacity.

### CONCLUSIONS

¹⁸FDG-PET plays an important role in the standard of care for cancer patients, but has limitations in prostate due to bladder contamination effects and difficulty distinguishing perfusion from metabolism. HP DHA provides a means to look at perfusion (the sum of VitC and DHA) as well as in vivo conversion (ratio of VitC/[VitC+DHA]). In the TRAMP model, increased reduction of HP [1-¹³C] DHA was observed in tumor, correlating with both elevated glucose uptake (as seen in ¹⁸F FDG studies), and high concentrations of GSH [7]. Since alterations in redox have been implicated in tumor aggressiveness and resistance to therapy, HP [1-¹³C] DHA could be used to predict and monitor treatment outcomes.

### Example 5

### Molecular Correlates to In Vivo Hyperpolarized [1-¹³C] Dehydroascorbate Reduction

### Introduction

Hyperpolarized (HP) ^{1[}1-¹³C] dehydroascorbate (DHA) is a recently developed probe that has demonstrated potential for in vivo imaging of reduction and oxidation (redox), which is highly dysregulated in cancer cells. ¹³C MRSI studies with HP DHA in a transgenic model of prostate cancer (TRAMP) model have shown rapid uptake of HP DHA into the cell via glucose (GLUT) transporters with subsequent reduction to ascorbate (vitamin C), from which the redox state of the tissue can be assessed. This redox state is also known to correlate with the concentration of glutathione (GSH) and non-protein thiols, which in conjunction with several intracellular enzymes including thioredoxin reductase, mediate reduction of DHA to vitamin C. High glutathione levels have been associated with cancer cell resistance to therapy, and clinical studies have found higher quantities of glutathione in tumors compared to normal tissue. Similarly, thioredoxin reductase has been shown to be elevated in certain human cancer cell lines and has been correlated with increased aggressiveness. We hypothesize that the observed reduction of HP [1-¹³C] DHA in vivo correlates with (1) uptake via GLUT-type transporters, (2) the intracellular GSH concentration, and (3) intracellular enzymes involved with the reduction of DHA. The purpose of this study is thus to correlate the high HP [VitC] signals observed by MRSI in the tumors of TRAMP mice with transporter expression, intracellular glutathione concentration, and enzymes involved in regulating redox.

### Methods

3 T MR Studies: In vivo MRSI studies using HP [1-¹³C] DHA were performed. Data Processing and Analysis: In vivo MRSI data was processed using custom software written in IDL 8 (ITT Visual Information Solutions, CO, USA) and Matlab 2009b (MathWorks, MA, USA). DHA and Vitamin C resonance integrals were used to calculate relevant ratios. Average metabolite ratios (VitC/[VitC+DHA]) were calculated for voxels corresponding to both tumor and surrounding benign tissues in TRAMP mice (n=3). Mercury Orange Staining and Fluorescent Microscopy: 8 μm tissue sections from TRAMP mice (n=3) and a normal mouse (n=1) were immersed in 50 μM mercury orange in toluene and rinsed with toluene after 4 minutes then mounted. Slides were viewed by dark-field, phase-contrast transmission fluorescence on a Nikon Eclipse Ti microscope (Nikon, N.Y., USA) with excitation frequency of 450-490 nm and emission filter barrier frequency of 600 nm. Mean fluorescent intensity was measured in 3 separate fields and averaged using NIS-Elements software (Nikon, N.Y., USA). GSH Measurement: Tissue samples were homogenized in PBS with 0.04% EDTA and assayed with a 5,5′-dithio-bis-2-(nitrobenzoic acid) based absorbance assay (Cayman, Mich., USA). Real-Time PCR: PCR primers were obtained from Applied Biosciences (Life Technologies, CA, USA), and real-time PCR was performed on total RNA isolated from frozen tissue extracts of TRAMP mice (n=3) and a normal mouse prostate cell line (n=1).

### Results and Discussion

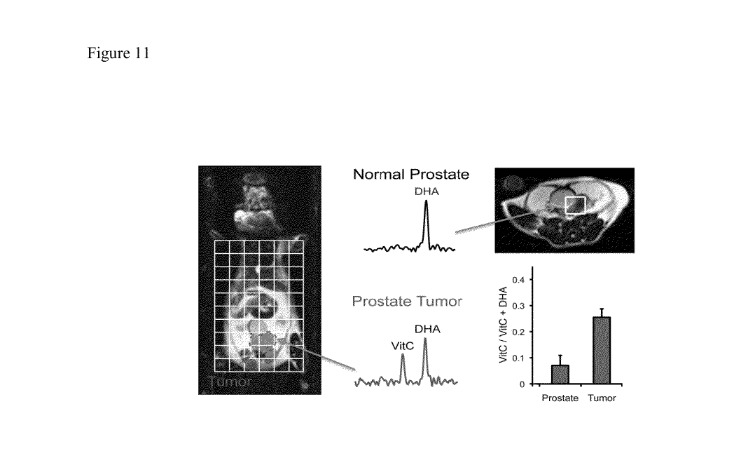

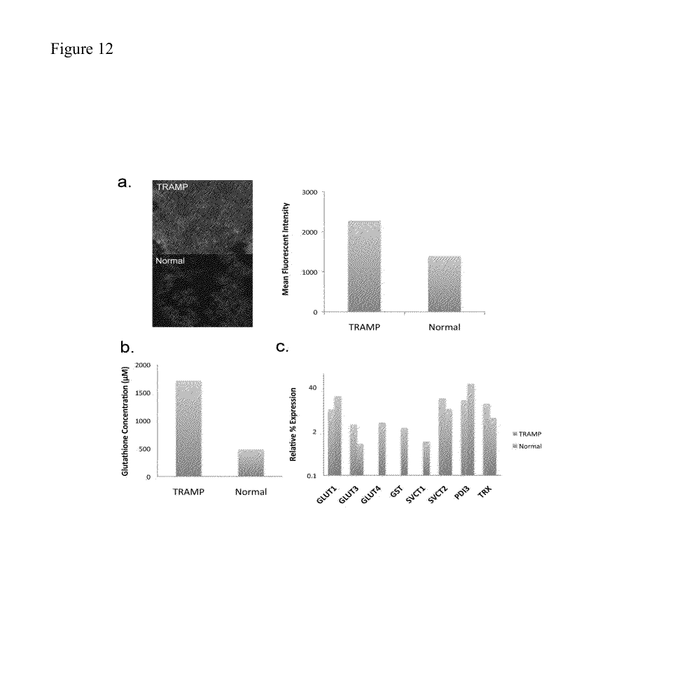

TRAMP tumors (n=3) demonstrated elevated ratios of HP vitamin C to DHA (VitC/[VitC+DHA]=0.23±0.03) compared to surrounding normal tissues and benign prostate (VitC/[VitC+DHA]=0.06±0.03) (FIG. 11). The level of non-protein thiols observed on mercury orange staining was elevated both qualitatively and quantitatively (FIG. 12a); mean fluorescent intensity averaged over three ROIs was 2284±265 in TRAMP tumors (n=3) versus 1394 in normal prostate (n=1). Intracellular glutathione levels were significantly elevated in TRAMP prostate (1707 μM±74, n=3) compared to normal prostate (481 μM, n=1) (FIG. 12b). The relative expression of GLUT1 and GLUT4 was decreased in TRAMP (8.7 and 0 relative % expression, respectively) compared to normal (21.2 and 3.6 relative % expression, respectively), but GLUT3 and thioredoxin reductase expression levels were increased (3.1 and 12.7 relative % expression, respectively, compared to 0.8 and 4.9) (FIG. 12c).

### CONCLUSIONS

The purpose of this study was to further elucidate the mechanism of HP¹³C DHA reduction observed in MRSI studies, with respect to transport, concentration of reducing agent (glutathione), and expression of enzymes that maintain intracellular redox. Surprisingly, analysis of GLUT transporter expression using RT-PCR revealed decreased GLUT1 and GLUT4 mRNA transcripts in TRAMP tumors, although GLUT3 mRNA transcripts were elevated. Future studies will focus on direct interrogation of GLUT transporter proteins (such as by Western Blotting) as mRNA transcripts may not accurately reflect membrane transporter density. Glutathione levels in TRAMP tumors were elevated both by mercury orange staining and by assay of homogenized tissue. This correlation of elevated HP vitamin C to DHA ratios in TRAMP tumors with elevated glutathione suggests that HP DHA MRSI is a noninvasive method to assess levels of GSH. This is significant because elevated GSH levels are correlated with therapeutic resistance in tumors. Finally, analysis of key redox proteins with RT-PCR revealed elevated thioredoxin reductase mRNA, which is significant given findings of elevated thioredoxin reductase in more aggressive lung cancer phenotypes.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A probe for in vivo MR imaging of a member selected from a first member, a second member and a combination thereof of an in vivo redox couple, said probe comprising:
(a) a molecular structure capable of reduction, oxidation or a combination thereof in an in vivo redox process as a covalently bound structural element;
(b) a greater than natural abundance of a hyperpolarizable atom, which, when hyperpolarized, is detectable in said MR imaging, wherein said atom is a covalently bound component of said molecular structure.

2. The probe according to claim 1 wherein said hyperpolarizable atom is a member selected from ¹³C and ¹⁵N.

3. The probe according to claim 1 wherein said hyperpolarizable atom is hyperpolarized.

4. The probe according to claim 1 wherein said probe is a component of a pharmaceutical formulation comprising said probe and a pharmaceutically acceptable diluent.

5. The probe according to claim 1 wherein said hyperpolarizable atom is hyperpolarized.

6. The probe according to claim 1 wherein said probe has a structure which is a member selected from:

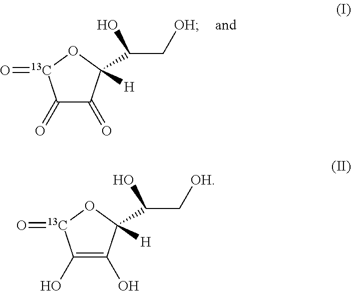 [SMILES: O=C1C(=O)[13C](=O)O[C@@H]1[C@@H](O)CO ; O=[13C]1O[C@H]([C@@H](O)CO)C(O)=C1O]

7. The probe according to claim 6 wherein said probe is further substituted by a member selected from:
(a) replacement of at least one ¹H atom with ²H;
(b) replacement of at least one ¹²C atom with ¹³C; and
(c) a combination thereof.

8. The probe according to claim 1 wherein said redox couple comprises:

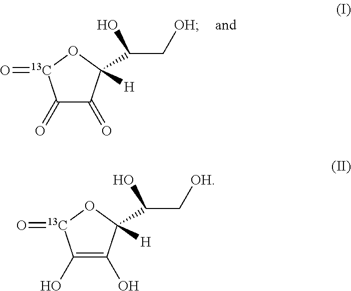 [SMILES: O=C1C(=O)[13C](=O)O[C@@H]1[C@@H](O)CO ; O=[13C]1O[C@H]([C@@H](O)CO)C(O)=C1O]

9. A method for in vivo MR imaging of a member selected from a first member, a second member and a combination thereof of an in vivo redox couple, said method comprising:
(a) administering a hyperpolarized, MR-detectable probe according to claim 5 to a subject to be imaged; and
(b) imaging a region of said subject containing said hyperpolarized probe in a manner appropriate to detect said member selected from said first member, said second member and said combination thereof of said in vivo redox couple.

10. The method according to claim 9, wherein said imaging is functional MR imaging.

11. A perfused system comprising a member selected from a cell, a tissue and a combination thereof, and a probe according to claim 1.

12. The perfused cell system according to claim 11 wherein said probe is hyperpolarized.

13. The perfused cell system according to claim 11, further comprising an agent of known or postulated therapeutic efficacy.

14. An in vitro enzyme assay system comprising a probe according to claim 1 and an enzyme to be assayed.

15. The enzyme assay system according to claim 14, wherein said enzyme is assayed for changes in redox potential.
